(19) United States
(12) Patent Application Publication
(10) Pub. No.: US 2011/0071323 A1
(43) Pub. Date: Mar. 24, 2011

(54) Method For Producing 1,2-Propanediol By Hydrogenolysis Of Glycerin

(75) Inventors: Achim Stankowiak, Altoetting (DE); Oliver Franke, Muenchen (DE); Joerg Appel, Tuessling (DE); Dirk Buehring, Burghausen (DE); Olaf Wachsen, Garching (DE)

(73) Assignee: CLARIANT FINANCE (BVI) LIMITED, Tortola (VG)

(21) Appl. No.: 12/993,923

(22) PCT Filed: May 27, 2009
(86) PCT No.: PCT/EP2009/003770

§ 371 Date: Nov. 22, 2010

(30) Foreign Application Priority Data
Jun. 10, 2008 (DE) 10 2008 027 601.4

(51) Int. Cl. C07C 31/18

(52) U.S. Cl. 568/852

(57) ABSTRACT

The invention relates to a method for producing 1,2-propanediol, in that a liquid phase comprising at least 95 wt.-% glycerin is reacted with hydrogen in the presence of a copper-containing, powdered catalyst in a continuously operated stirred reactor at a pressure of 50 to 90 bar, wherein the stationary yield of the reaction is at least 60%.

The present invention relates to a method for producing 1,2-propanediol from glycerin in the continuous slurry method.

1,2-Propanediol is currently produced on an industrial scale from propylene oxide by the addition reaction of water and is used in a large number of applications, such as e.g. as a constituent of braking and hydraulic fluids, lubricants and antifreezes, in cosmetics, in the food industry, and as solvents for fats, oils, resins and dyes. Propylene oxide and thus also 1,2-propanediol is currently still produced completely from fossil fuels. On account of the continuing demand for using renewable raw materials, and the increasing cost of crude oil and falling cost of glycerin, there is a great need to use glycerin, which is produced as a by-product in large amounts in the production of biodiesel, as a starting material for suitable chemical reactions on a large scale in industry.

The catalytic hydrogenation of glycerin to 1,2-propanediol has already frequently been investigated.

DE-A-44 42 124 describes the catalytic hydrogenation of glycerin with a water content of up to 20% by weight to give propylene glycol in a yield of 92%, n-propanol and lower alcohols being obtained as by-products. The complete conversion of glycerol is achieved through the use of a mixed catalyst of the metals cobalt, copper, manganese and molybdenum. The reaction is carried out in an autoclave or in a trickle reactor. The reaction conditions are in a pressure and temperature range from 100 to 700 bar and 180 to 270° C. Preferred hydrogenation conditions are 200 to 325 bar and 200 to 250° C. It is disadvantageous that at relatively low pressures the reaction of the glycerin is incomplete, and at relatively high pressures, lower alcohols are formed to an increased extent.

U.S. Pat. No. 4,642,394 describes a process for the catalytic hydrogenation of glycerin with a catalyst consisting of tungsten and a group VIII metal. The reaction conditions are in the range from 100 psi to 15 000 psi and 75 to 250° C. Preferred process conditions are 100 to 200° C. and 200 to 10 000 psi. The reaction is carried out under basic conditions through the use of amines or amides as solvents. It is also possible to use metal hydroxides, metal carbonates or quaternary ammonium salts. The solvent is added in a concentration of from 5 to 100 ml per gram of glycerin. Carbon monoxide is used for the stabilization and activation of the catalyst.

EP-A-0 523 015 describes the hydrogenation of glycerin over Cu/Zn catalysts, but working with very dilute aqueous solutions (ca. 30% by weight glycerin content) which become yet further diluted as a result of the water of reaction which is formed. Consequently, in order to obtain 1,2-propanediol, a large amount of water has to be distilled off from the product, which signifies a high energy expenditure and makes the method uneconomical. Moreover, the method is carried out at relatively high pressures of preferably 100-150 bar and high temperatures of 230-270° C. in autoclaves or tubular reactors. The conversion of glycerin is in the range from 8 to 100% at a selectivity to propylene glycol of from 80 to 98%, and alcohols and ethylene glycol are formed as by-products

DE-A-43 02 464 describes a method in which glycerin is hydrogenated in a continuous procedure over a CuO/ZnO fixed-bed catalyst. In this method, a complete hydrogenation of glycerin is achieved at 200° C.; the by-products formed are small amounts of low-hydricity alcohols and relatively large amounts (5.4% by weight) of unknown substances. A disadvantage is likewise the very high reaction pressure of 250 bar. At relatively low pressures (50-150 bar) and relatively high temperatures (240° C.) unknown substances (25-34% by weight) are formed to an increased extent, with the selectivity to 1,2-propanediol falling to 22-31% by weight.

WO 2007/10299 describes a method in which glycerin is hydrogenated in a hydrogen-containing gas stream in a continuous procedure in the vapor phase in the presence of a copper catalyst. The reaction temperature is between 160 and 260° C., the reaction pressure between 10 and 30 bar. The conversion of glycerin is in the range from 97 to 100% with a selectivity to propylene glycol of from 93 to 96%; alcohols and ethylene glycol are formed as by-products. A disadvantage of the method is the low space-time yield.

US 2005/0244312 A1 describes a process for the hydrogenation of glycerin to 1,2-propanediol via hydroxyacetone as intermediate at a temperature of from 150 to 250° C. and a pressure of from 1 to 25 bar. A preferred catalyst for this reaction is copper chromite. At 200° C. and 14 bar, the conversion after 24 hours is 55% with a selectivity of 80%.

It has now been found that during the hydrogenation of glycerin to 1,2-propanediol, even with other copper-containing catalysts, such as e.g. Raney copper or CuO/ZnO, hydroxyacetone is formed as intermediate, which decomposes in an extremely exothermic reaction if insufficient hydrogen is available for a further hydrogenation to 1,2-propanediol. This decomposition can take place in an explosive fashion and can compromise the operating safety of a corresponding production apparatus. It is therefore an object of the present invention to find a method in which the steady-state concentration of hydroxacetone remains below the critical limit and produces the 1,2-propanediol in high selectivity and high space-time yield.

Surprisingly, it has been found that such a method has been found in the hydrogenation of essentially pure glycerin in liquid phase with powder catalysis if its steady-state conversion is at least 60%.

The invention therefore provides a method for producing 1,2-propanediol by reacting a liquid phase which comprises at least 95% by weight of glycerin with hydrogen in the presence of a copper-containing, pulverulent catalyst in a continuously operated stirred reactor at a pressure of from 50 to 90 bar, where the steady-state conversion of the reaction is at least 60%.

A steady-state conversion of 100% means in this case complete conversion of the glycerin.

In the method according to the invention, the catalyst is preferably separated from the reaction mixture under the reaction conditions via a sedimentation or crossflow filtration and returned to the reactor. A separating-off of the catalyst under reaction conditions is a preferred constituent of the method according to the invention since only in this way is the high activity and selectivity of the catalyst maintained.

The reactor is operated at a steady-state conversion of preferably 60 to 95%, in particular 65 to 85%, the unreacted glycerin preferably being recovered after separating off the catalyst by distillative work-up. An advantage of the method according to the invention over the prior art is that no process hydrogen has to be circulated because the volume-proportionate consumption of the hydrogen corresponds to the stoichiometry of the reaction and does not have to be supplied to the reactor in a substantially higher proportion and be recycled again.

The catalysts used in the method according to the invention are copper-containing catalysts, such as e.g. Raney copper, copper chromite or copper oxide-zinc oxide.

The hydrogenation of glycerin is carried out in the method according to the invention at temperatures of preferably 180-240, in particular 200-220° C.

The hydrogenation of glycerin is carried out in the method according to the invention at pressures of preferably 60-80 bar.

If crude glycerin from the transesterification of fats and oils is used in the method according to the invention, it should expediently be concentrated by distillation and be freed from catalyst poisons, such as sulfuric acid, which is often used as a transesterification catalyst.

The method according to the invention has the advantage that the desired reaction product 1,2-propanediol is formed with the catalyst used in high selectivity of up to 97%. The only by-products that can be detected are monoethylene glycol and small amounts of alcohols such as n-propanol, isopropanol and ethanol, which can be removed easily by distillation with the water of reaction. The product mixture obtained can, if required, either be used directly for chemical applications or be converted to pure 1,2-propanediol (>99.5% by weight) by further distillative purification.

The following examples illustrate the invention:

### EXAMPLE 1

### Method for Producing 1,2-propanediol by Hydrogenolysis of Glycerin over a CuO/ZnO Catalyst

In a continuous-flow stirred-tank reactor with a volume of 10 l, a reactor fill level of ca. 70% and a catalyst loading of 4% by weight, 0.5 kg/h of glycerin were metered in at a reactor internal temperature between 200 and 210° C. The pressure in the reactor was 80 bar, which was kept constant on the offgas side via a pressure relief valve. A stream of ca. 100 l/h (STP) of hydrogen was passed into the reactor. Samples from the reactor overflow revealed a steady-state conversion of glycerin of 84.5% with a product selectivity of 95.6%. The mass fraction of glycerin was 15.12% for this procedure.

### EXAMPLE 2

### Method for Producing 1,2-propanediol by Hydrogenolysis of Glycerin over a CuO/ZnO Catalyst

In a second example, in the same equipment as in example 1, a higher throughput of ca. 1.42 kg/h of glycerin was used. The pressure and the temperature were kept in the same range as in example 1. A steady-state conversion of glycerin of 66.24% was observed. At this conversion, the mass fraction of glycerin was approximately 33%, which, according to the safety considerations, can be classed as safe.

Estimation of the temperature increases that arise as a function of the steady-state conversion in the method according to the invention.

From safety analyses by means of DTA measurements of the chemical compounds involved in the reaction in the presence of the catalyst, it was possible to ascertain the decomposition energy of glycerin and the temperature at the start of decomposition. It was found that the decomposition of glycerin in the presence of the CuO/ZnO catalyst proceeds with release of energy of ca. 450 kJ/kg. 1,2-Propylene glycol is stable under the same conditions. In a simple estimation, the increase in the temperature during the decomposition of glycerin in a mixture of glycerin and propylene glycol can be ascertained. The specific thermal capacity at a constant pressure cₚ of glycerin at 180 to 2500° C. is between 2670 and 2940 J/kg/K. Under the same conditions, 1,2-propylene glycol has a cₚ of 3050 to 3770 J/kg/K. The estimation was carried out with the lowest values for cₚ in order to obtain a maximum adiabatic temperature increase.

If now the case of 66% conversion (example 2) with a glycerin fraction of 33% is assumed, then in an adiabatic reactor upon complete decomposition of the glycerin, an increase in the temperature of ca. 50 K would be expected. In the case of example 1 with 84.5% conversion, the increase is ca. 23 K.

The risk due to the specified increases in temperature is considerably lower than for a batchwise operation of the reactor, in which exclusively glycerin is present at the start of the reaction. Here, an increase of more than 150 K would have to be reckoned with.

1. A method for producing 1,2-propanediol by reacting a liquid phase which comprises at least 95% by weight of glycerin with hydrogen in the presence of a copper-containing, pulverulent catalyst in a continuously operated stirred reactor at a pressure of from 50 to 90 bar, where the steady-state conversion of the reaction is at least 60%.

2. The method as claimed in claim 1, in which the catalyst is separated from the reaction mixture under the reaction conditions via a sedimentation or crossflow filtration and returned to the reactor.

3. The method as claimed in claim 1 and/or 2, in which the reactor is operated at a steady-state conversion of 65 to 85%, the unreacted glycerin being recovered after separating off the catalyst by distillative work-up.

4. The method as claimed in one or more of claims 1 to 3, wherein Raney copper, copper chromite or copper oxide-zinc oxide are used as copper-containing catalysts.

5. The method as claimed in one or more of claims 1 to 4, wherein the hydrogenation of glycerin is carried out at temperatures of 200-220° C.
